(19) United States
(12) Patent Application Publication
(10) Pub. No.: US 2021/0188727 A1
(43) Pub. Date: Jun. 24, 2021

(54) AMINO ACID FERTILIZER COMPOSITION MANUFACTURED FROM LIVESTOCK FARMING BY-PRODUCT USING ACID, ALKALI AND PROTEASE TREATMENT

(71) Applicant: Liren Bio Co., Ltd., Gyeongsangnam-do (KR)

(72) Inventor: Bong Ki KIM, Gyeongsangnam-do (KR)

(21) Appl. No.: 16/092,253

(22) PCT Filed: May 3, 2018
(86) PCT No.: PCT/KR2018/005122

§ 371 Date: Oct. 9, 2018

(30) Foreign Application Priority Data
Jun. 1, 2017 (KR) 10-2017-0068222

(51) Int. Cl. C05F 1/00; C05F 17/20; C05F 17/10; C12N 15/01; A01G 22/05; A01G 22/15

(52) U.S. Cl. CPC C05F 1/005; C05F 17/20; A01G 22/15; C12N 15/01; A01G 22/05; C05F 17/10

(57) ABSTRACT

An amino acid fertilizer composition manufactured from livestock farming by-product using acid, alkali and protease treatment. More specifically, the amino acid fertilizer composition may be manufactured by: 1) separating and producing a crude collagen peptide from livestock farming by-product, such as, bovine skin, pig skin or bovine bone using acid and alkali treatment, 2) hydrolyzing the obtained crude collagen peptide using collagen specific protease produced from Bacillus sp. variant Iren-101, and 3) obtaining an amino acid level fertilizer composition.

### TECHNICAL FIELD

The present invention relates to an amino acid fertilizer composition manufactured from livestock farming by-product using acid, alkali and protease treatment. More specifically, this invention relates to an amino acid fertilizer composition manufactured by the steps comprising: 1) separating and producing a crude collagen peptide from livestock farming by-product, such as, bovine skin, pig skin or bovine bone using acid and alkali treatment, 2) hydrolyzing the obtained crude collagen peptide using collagen specific protease produced from Bacillus sp. variant Iren-101, and 3) obtaining an amino acid level fertilizer composition.

### DESCRIPTION OF PRIOR ART

Recently, an amino acid fertilizer has been marketed for supplying the sufficient amino acid nutrition to crops. Further, it has been regarded as effective product for promoting the growth of crops by enhancing the nitrogen efficiency.

Even if plant can produce its own amino acids in itself, plant sometimes requires external amino acids for converting or storing them as protein for the use of various kinds of physiologically active substances or metabolic energy sources.

On the other hand, if the amino acid fertilizer is supplied to the crops, the amino acid fertilizer can act as a nutrient source of soil microbes, which can cause the growth of soil microbes as well as the promotion of root activity of crops.

Since the amino acid fertilizer consists of organic amino acid compound, it can be easily biosynthesized into protein in the crops rather than nitrogen fertilizer as inorganic salt form. Of course, it can show high growth promotion effect to the crops.

In Korean Patent No. 10-1341721 “Process for preparing an amino acid fertilizer composition from fish byproduct”, the amino acid fertilizer from fish byproduct has been disclosed. Moreover, it has been disclosed that the amino acid fertilizer has been manufactured by fish collagen peptide after hydrolysis of fish skin byproduct.

On the other hand, the amino acid fertilizer disclosed in this patent disclosure may be chiefly present in the level of fish collagen peptide, because the hydrolysis from fish collagen peptide to amino acid level has not been specifically disclosed.

Since only acid or alkali hydrolysis treatment of fish byproduct cannot decompose the cross linkage in the collagen protein, such as, disulfide bond, the hydrolysis to the amino acid level cannot be fulfilled without further hydrolysis treatment. Moreover, the hydrolysis to amino acid level hardly is carried out using ordinary protein hydrolysis enzyme, such as, pepsin or trypsin.

Therefore, the inventor of present invention has tried to develop an efficient amino acid fertilizer composition wherein the collagen protein is decomposed into the amino acid level using collagen specific protease produced from Bacillus sp. variant Iren-101.

Finally, the present invention has completed by 1) separating and producing a crude collagen peptide from livestock farming by-product, such as, bovine skin, pig skin or bovine bone using acid and alkali treatment, 2) hydrolyzing the obtained crude collagen peptide using collagen specific protease produced from Bacillus sp. variant Iren-101, and 3) obtaining an amino acid level fertilizer composition.

### PROBLEM TO BE SOLVED

The problem to be solved is to develop an amino acid fertilizer composition wherein the collagen protein is decomposed into the amino acid level using collagen specific protease produced from Bacillus sp. variant Iren-101. Further, it is also intend to develop an efficient amino acid fertilizer composition manufactured by 1) separating and producing a crude collagen peptide from livestock farming by-product, such as, bovine skin, pig skin or bovine bone using acid and alkali treatment, 2) hydrolyzing the obtained crude collagen peptide using collagen specific protease produced from Bacillus sp. variant Iren-101, and 3) obtaining an

### MEANS FOR SOLVING THE PROBLEM

The object of present invention is to provide an amino acid fertilizer composition manufactured by the steps comprising:

1) separating and producing a crude collagen peptide solution by the hydrolysis of protein from 100 wt part of dried and crushed bovine skin, pig skin or bovine bone using both pH 1.0-3.0 acid treatment and pH 12.0-14.0 alkali treatment;

2) hydrolyzing and decomposing the obtained crude collagen peptide into the amino acid level using both 0.01-0.5 wt part of collagen specific protease produced from Bacillus sp. variant Iren-101 and 0.001-0.1 wt part at least one protease selected from serine endo-protease or exo-peptidase; and

3) obtaining a liquid phase amino acid fertilizer by adding 0.001-0.1 wt part of at least one water soluble inorganic salt selected from potassium, calcium, magnesium, iron or phosphate to the neutralized amino acid solution, after neutralizing the obtained amino acid solution in step 2).

Further, the total amount of amino acid in said fertilizer composition is 38-50 wt %, and major amino acid amount includes 8-11 wt % of glycine, 5.0-7.0 wt % of proline, 3.5-5.0 wt % of alanine, 3.0-4.5 wt % of gultamic acid, 2.5-3.5 wt % of arginine, 1.8-2.5 wt % of aspartic acid, 2.5-3.5 wt % of leucine, 1.0-1.5 wt % of valine and 1.0-1.5 wt % of isoleucine of total fertilizer composition.

Further, said Bacillus sp. variant Iren-101 can be obtained by following isolating steps comprising 1) selecting the strain of Bacillus sp. alive in soil showing the highest collagen assimilation property, 2) mutating the gene of strain with N-Methyl-N′-nitro-N-nitrosoguanidine (NTG) for selecting the variant showing the highest collagen assimilation property, and 3) isolating the Bacillus sp. variant Iren-101 after more than 3 times subculturing.

On the other hand, the strawberry administered with 30-100 ppm of said amino acid fertilizer composition increases 20-30% of the number of the fruit, 5-10% of the weight of the fruit, 10-20% of the volume of the fruit and 5-10% of the sugar content of the fruit compared to those of non-administered strawberry.

Further, the lettuce administered with 5-50 ppm of said amino acid fertilizer composition increases 25-40% of the leaf length, 10-20% of the leaf width and 20-30% of the chlorophyll index compared to those of non-administered lettuce.

In addition, aspartic acid and glutamic acid in said amino acid fertilizer composition can be effective for overcoming the physiological disorder caused by cold weather and lack of sunshine to the crops, while glycine, alanine and leucine can increase the sugar content of crops and arginine can improve the pathogen microbe resistance of the crops.

### ADVANTAGEOUS EFFECT

The outstanding advantageous effect of the present invention is to provide an amino acid fertilizer composition wherein the collagen protein is decomposed into the amino acid level using collagen specific protease produced from Bacillus sp. variant Iren-101. Further, it also provide an efficient amino acid fertilizer composition manufactured by 1) separating and producing a crude collagen peptide from livestock farming by-product, such as, bovine skin, pig skin or bovine bone using acid and alkali treatment, 2) hydrolyzing the obtained crude collagen peptide using collagen specific protease produced from Bacillus sp. variant Iren-101, and 3) obtaining an amino acid level fertilizer composition.

### BRIEF DESCRIPTION OF DRAWINGS

FIG. 1 shows HPLC data indicating the amino acid distribution in the amino acid fertilizer prepared in Preparation Example 1 of the present invention. The Agilent HPLC has been used. The column is a ZORBAX Eclipse-AAA column. The eluent A is 40 mM phosphate buffer. The eluent B is a mixed solvent of methanol/acetonitrile/water=45/45/10. An alanine peak has been detected near the retention time of 10 minutes and glycine, glutamic acid and valine peaks have been detected between 10 minutes and 12.5 minutes. In addition, leucine peak has been detected at the retention time of 12.5 minutes. Further, aspartic acid and proline peaks have been detected at 13 minutes and 13.5 minutes respectively.

### PREFERRED EMBODIMENT OF INVENTION

The present invention relates to an amino acid fertilizer composition manufactured by the steps comprising:

1) separating and producing a crude collagen peptide solution by the hydrolysis of protein from 100 wt part of dried and crushed bovine skin, pig skin or bovine bone using both pH 1.0-3.0 acid treatment and pH 12.0-14.0 alkali treatment;

2) hydrolyzing and decomposing the obtained crude collagen peptide into the amino acid level using both 0.01-0.5 wt part of collagen specific protease produced from Bacillus sp. variant Iren-101 and 0.001-0.1 wt part at least one protease selected from serine endo-protease or exo-peptidase; and

3) obtaining a liquid phase amino acid fertilizer by adding 0.001-0.1 wt part of at least one water soluble inorganic salt selected from potassium, calcium, magnesium, iron or phosphate to the neutralized amino acid solution, after neutralizing the obtained amino acid solution in step 2).

Further, the total amount of amino acid in said fertilizer composition is 38-50 wt %, and major amino acid amount includes 8-11 wt % of glycine, 5.0-7.0 wt % of proline, 3.5-5.0 wt % of alanine, 3.0-4.5 wt % of gultamic acid, 2.5-3.5 wt % of arginine, 1.8-2.5 wt % of aspartic acid, 2.5-3.5 wt % of leucine, 1.0-1.5 wt % of valine and 1.0-1.5 wt % of isoleucine of total fertilizer composition.

On the other hand, the strawberry administered with 30-100 ppm of said amino acid fertilizer composition increases 20-30% of the number of the fruit, 5-10% of the weight of the fruit, 10-20% of the volume of the fruit and 5-10% of the sugar content of the fruit compared to those of non-administered strawberry.

Further, the lettuce administered with 5-50 ppm of said amino acid fertilizer composition increases 25-40% of the leaf length, 10-20% of the leaf width and 20-30% of the chlorophyll index compared to those of non-administered lettuce.

The present invention can be explained more specifically as follows.

The preparation steps of an amino acid fertilizer composition of the present invention can be explained in detail as follows.

(Step 1) Step for Separating and Producing a Crude Collagen Peptide Solution

This preparation step is to separate and produce a crude collagen peptide solution by the hydrolysis of protein from 100 wt part of dried and crushed bovine skin, pig skin or bovine bone using both pH 1.0-3.0 acid treatment and pH 12.0-14.0 alkali treatment.

After 200-500 wt part of purified water is added to 100 wt part of bovine skin, pig skin or bovine bone, the mixture is treated with both pH 1.0-3.0 of HCl solution for 18-30 hours and pH 12.0-14.0 of sodium hydroxide for 18-30 hours for hydrolyzing and decomposing proteins and lipids. Upon removing the solid residue materials in the reactor, a crude collagen peptide solution can be obtained.

(Step 2) Step for Obtaining Amino Acid Solution

This preparation step is to obtain an amino acid solution by hydrolyzing and decomposing the obtained crude collagen peptide in Step 1 into the amino acid level using both 0.01-0.5 wt part of collagen specific protease produced from Bacillus sp. variant Iren-101 and 0.001-0.1 wt part at least one protease selected from serine endo-protease or exo-peptidase.

In this step, Bacillus sp. variant Iren-101 has been prepared by following isolation steps comprising 1) selecting the strain of Bacillus sp. alive in soil showing the highest collagen assimilation property, 2) mutating the gene of strain with N-Methyl-N′-nitro-N-nitrosoguanidine (NTG) for selecting the variant showing the highest collagen assimilation property, and 3) isolating the Bacillus sp. variant Iren-101 after more than 3 times subculturing. Since Bacillus sp. variant Iren-101 can use the collagen as energy source, it means that this variant can produce the collagen specific protease for effectively hydrolyzing collagen proteins.

Therefore, the crude collagen peptide is hydrolyzed and decomposed into amino acid level using both 0.01-0.5 wt part of collagen specific protease produced from Bacillus sp. variant Iren-101 and 0.001-0.1 wt part at least one protease selected from serine endo-protease or exo-peptidase.

The preferred serine endo-protease can be ‘Alcalase’ commercially available from Novozymes, while the preferred exo-peptidase can be ‘Flavourzyme’ commercially available from Novozymes. The preferred amount of these serine endo-protease and exo-peptidase can be ⅕ weight amount of collagen specific protease produced from Bacillus sp. variant Iren-101.

(Step 3) Step for Obtaining an Amino Acid Fertilizer

This preparation step is to obtain a liquid phase amino acid fertilizer by adding 0.001-0.1 wt part of at least one water soluble inorganic salt selected from potassium, calcium, magnesium, iron or phosphate to the neutralized amino acid solution, after neutralizing the obtained amino acid solution in step 2.

The preferred pH of amino acid fertilizer is 6.5-9.0, which can effectively prevent the soil acidification as well as restore the soils with backflow obstruction. Since the amino acid solution prepared in Step 2 exhibits a slightly acidic pH (pH 5.0-6.5), the amino acid solution is required to be neutralized to pH 6.5-9.0 by adding a weakly alkaline base.

Further, by adding at least one water soluble inorganic salt selected from potassium, calcium, magnesium, iron or phosphate to the amino acid solution, the more useful amino acid fertilizer for plant growth can be provided.

Since the amino acid fertilizer composition prepared in the present invention is completely separated into amino acid level by specific enzyme hydrolysis, the amino acid fertilizer composition can be present in a liquid phase at room temperature, even though it contains the high amino acid concentration of 38-50 wt %.

The isolation method of Bacillus sp. variant Iren-101 and collagen specific protease produced from this variant can be explained in detail as follows.

In order to select strains producing collagen specific protease, Bacillus sp. strains are collected from soil at Changwon city in Korea. After diluting the collected strains with sterilized water, the diluted strains are plated on the skim milk agar medium containing 20 wt % of collagen (5 g/L of collagen-containing skim milk, 10 g/L of bacto tryptone, 5 g/L of yeast extract, 5 g/L of NaCl and 5 g/L of bacto agar).

After incubating the strains on said medium for more than 15 hours in the 30° C. incubator, growing and producing Bacillus sp. colony is primarily selected from the relatively large size of clear zone according to the decomposition of proteins in the collagen-containing skim milk.

Upon mutating the Bacillus sp. strain by N-Methyl-N′-nitro-N-nitrosoguanidine (NTG), the Bacillus sp. variant is selected from the colony showing the largest size of clear zone. Finally, Bacillus sp. variant Iren-101 is isolated after 3 times subculturing.

After incubating the strains on the LB medium, which contains one of nitrogen source, such as, malt extract, yeast extract, casein or collagen, the growth of strain and the protease activity of Bacillus sp. variant Iren-101 to each nitrogen source have been measured with stirring at 150 rpm for 15 hours at 30 C. The results are shown in Table 1.

TABLE 1

| | Growth of strain | Protease activity |
|---|---|---|
| Nitrogen source | (absorb. at 660 nm) | (U/ml) |
| Malt extract | 2.0 ± 0.2 | 40.4 ± 2.4 |
| Yeast extract | 2.7 ± 0.5 | 84.5 ± 3.2 |
| Casein | 2.3 ± 0.4 | 62.9 ± 3.1 |
| Collagen | 2.7 ± 1.5 | 90.4 ± 3.5 |

As shown in Table 1, the Bacillus sp. variant Iren-101 shows the high protease activity to the collagen protein. Therefore, it has been proved that Bacillus sp. variant Iren-101 is a collagen specific protease.

The advantageous effect of the amino acid fertilizer composition according to the present invention shows that the crops administered with the amino acid fertilizer composition increase 25-40% of the leaf length, 10-20% of the leaf width and 20-30% of the chlorophyll index compared to those of non-administered crops. Further, overall yields of crops increase more than 30 wt % compared to those of non-administered crops.

Also, the role of each amino acid in fertilizer composition has been examined Aspartic acid and glutamic acid in fertilizer composition can be effective for overcoming the physiological disorder caused by cold weather and lack of sunshine to the crops, while glycine, alanine and leucine can increase the sugar content of crops and arginine can improve the pathogen microbe resistance of the crops.

The present invention can be explained more concretely by following Preparation Example, Comparative Preparation Example and Example. However, it should be understood that Example is intended to illustrate but not in any manner to limit the scope of the present invention.

### (PREPARATION EXAMPLE 1)PREPARATION OF AMINO ACID FERTILIZER COMPOSITION OF THE PRESENT INVENTION

After 3L of purified water is added to 1 Kg of bovine skin, pig skin or bovine bone, the mixture is treated with both pH 1.5 of HCl solution for 24 hours and pH 13.5 of sodium hydroxide for 24 hours for hydrolyzing and decomposing proteins and lipids. Upon removing the solid residue materials in the reactor, 500 ml of crude collagen peptide solution (collagen content: 15 wt %) is obtained.

500 ml of obtained crude collagen peptide solution is hydrolyzed and decomposed into amino acid level using 1 g of collagen specific protease produced from Bacillus sp. variant Iren-101, 0.1 g of serine endo-protease (Trade name: Alcalase) (potency: 2.5 AU-A/g) and 0.1 g of exo-peptidase (Trade name: Flavourzyme) (potency: 500 LAPU/g). Then, 510 ml of amino acid solution is obtained.

Then, the obtained amino acid solution is neutralized into pH 8.0. Finally, 214 g of liquid phase amino acid fertilizer is obtained after adding 0.1 g of at least one water soluble inorganic salt selected from potassium, calcium, magnesium, iron or phosphate.

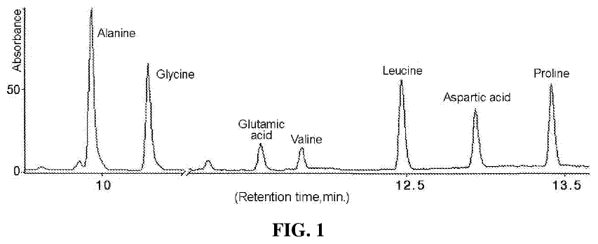

FIG. 1 shows HPLC data indicating the amino acid distribution in the amino acid fertilizer prepared in Preparation Example 1 of the present invention.

The Agilent HPLC has been used. The column is a ZORBAX Eclipse-AAA column. The eluent A is 40 mM phosphate buffer. The eluent B is a mixed solvent of methanol/acetonitrile/water=45/45/10. An alanine peak has been detected near the retention time of 10 minutes and glycine, glutamic acid and valine peaks have been detected between 10 minutes and 12.5 minutes. In addition, leucine peak has been detected at the retention time of 12.5 minutes. Further, aspartic acid and proline peaks have been detected at 13 minutes and 13.5 minutes respectively.

Through HPLC, it has been measured that the total amount of amino acid in said fertilizer composition is 43.3 wt % of fertilizer composition, and that major amino acid amount includes 9.7 wt % of glycine, 5.8 wt % of proline, 4.0 wt % of alanine, 3.8 wt % of gultamic acid, 2.7 wt % of arginine, 2.1 wt % of aspartic acid, 2.8 wt % of leucine, 1.3 wt % of valine and 1.1 wt % of isoleucine of total fertilizer composition.

### (COMPARATIVE PREPARATION EXAMPLE 1)PREPARATION OF AMINO ACID FERTILIZER COMPOSITION (WITHOUT TREATMENT OF COLLAGEN SPECIFIC PROTEASE)

After 3L of purified water is added to 1 Kg of bovine skin, pig skin or bovine bone, the mixture is treated with both pH 1.5 of HCl solution for 24 hours and pH 13.5 of sodium hydroxide for 24 hours for hydrolyzing and decomposing proteins and lipids. Upon removing the solid residue materials in the reactor, 500 ml of crude collagen peptide solution (collagen content: 15 wt %) is obtained.

500 ml of obtained crude collagen peptide solution is hydrolyzed and decomposed into amino acid level using 0.1 g of serine endo-protease (Trade name: Alcalase) (potency: 2.5 AU-A/g) and 0.1 g of exo-peptidase (Trade name: Flavourzyme) (potency: 500 LAPU/g). Then, 507 ml of amino acid solution is obtained.

Then, the obtained amino acid solution is neutralized into pH 8.0. Finally, 227 g of liquid phase amino acid fertilizer is obtained after adding 0.1 g of at least one water soluble inorganic salt selected from potassium, calcium, magnesium, iron or phosphate.

Through HPLC, it has been measured that the total amount of amino acid in said fertilizer composition is 34 wt % of fertilizer composition, and that major amino acid amount includes 8.0 wt % of glycine, 4.6 wt % of proline, 3.0 wt % of alanine, 2.5 wt % of gultamic acid, 2.5 wt % of arginine, 1.9 wt % of aspartic acid, 2.7 wt % of leucine, 1.3 wt % of valine and 1.0 wt % of isoleucine of total fertilizer composition.

### (COMPARATIVE PREPARATION EXAMPLE 2)PREPARATION OF AMINO ACID FERTILIZER COMPOSITION (WITHOUT TREATMENT OF SERINE ENDO-PROTEASE AND EXO-PEPTIDASE)

After 3L of purified water is added to 1 Kg of bovine skin, pig skin or bovine bone, the mixture is treated with both pH 1.5 of HCl solution for 24 hours and pH 13.5 of sodium hydroxide for 24 hours for hydrolyzing and decomposing proteins and lipids. Upon removing the solid residue materials in the reactor, 500 ml of crude collagen peptide solution (collagen content: 15 wt %) is obtained.

500 ml of obtained crude collagen peptide solution is hydrolyzed and decomposed into amino acid level using 1 g of collagen specific protease produced from Bacillus sp. variant Iren-101. Then, 498 ml of amino acid solution is obtained.

Then, the obtained amino acid solution is neutralized into pH 8.0. Finally, 194 g of liquid phase amino acid fertilizer is obtained after adding 0.1 g of at least one water soluble inorganic salt selected from potassium, calcium, magnesium, iron or phosphate.

Through HPLC, it has been measured that the total amount of amino acid in said fertilizer composition is 27 wt % of fertilizer composition, and that major amino acid amount includes 6.5 wt % of glycine, 4.5 wt % of proline, 2.0 wt % of alanine, 2.0 wt % of gultamic acid, 1.5 wt % of arginine, 1.9 wt % of aspartic acid, 1.8 wt % of leucine, 1.2 wt % of valine and 0.9 wt % of isoleucine of total fertilizer composition.

### (EXAMPLE 1)COMPARISON TEST OF STRAWBERRY YIELD

The amino acid fertilizer compositions prepared in Preparation Example 1, Comparative Preparation Examples 1 and 2 have been formulated as spraying solutions. Each 0.1 ml of solution per 1 strawberry has been sprayed to total 100 strawberries. 100 strawberries without administering any of amino acid fertilizer composition have been used as control group. Table 2 shows the number, weight, volume and sugar content of matured strawberries after administering each fertilizer composition.

TABLE 2

| | Number of | Weight of | Volume of | Sugar |
|---|---|---|---|---|
| | fruit | fruit | fruit | content |
| | (pieces) | (g/piece) | (ml/piece) | (Brix %) |
| Pre. Ex. 1 | 32.0 | 41.1 | 32.8 | 11.1 |
| Com. Pre. Ex. 1 | 28.4 | 38.5 | 30.2 | 10.7 |
| Com. Pre. Ex. 2 | 27.4 | 37.6 | 29.9 | 10.5 |
| Control | 25.3 | 38.1 | 28.5 | 10.2 |

As shown in Table 2, the strawberry administered with amino acid fertilizer composition of the present invention increases 26% of the number of the fruit, 7.8% of the weight of the fruit, 15% of the volume of the fruit and 8.8% of the sugar content of the fruit compared to those of non-administered strawberry.

### EXAMPLE 2 COMPARISON TEST OF LETTUCE YIELD

The amino acid fertilizer compositions prepared in Preparation Example 1, Comparative Preparation Examples 1 and 2 have been formulated as spraying solutions. Each 0.01 ml of solution per 1 lettuce has been sprayed to total 100 lettuces. 100 lettuces without administering any of amino acid fertilizer composition have been used as control group. Table 2 shows the leaf length, leaf width and chlorophyll index of matured lettuce after administering each fertilizer composition.

TABLE 3

| | Leaf length | Leaf width | Chloroph. Index |
|---|---|---|---|
| | (cm) | (cm) | (g/100 cm²) |
| Pre. Ex. 1 | 23.0 | 18.6 | 2.73 |
| Com. Pre. Ex. 1 | 21.8 | 16.8 | 2.45 |
| Com. Pre. Ex. 2 | 19.5 | 17.4 | 2.38 |
| Control | 16.9 | 16.1 | 2.21 |

As shown in Table 3, the lettuce administered with amino acid fertilizer composition of the present invention increases 36% of the leaf length, 15.5% of the leaf width and 23.5% of the chlorophyll index compared to those of non-administered lettuce.

As a conclusion, the amino acid fertilizer of the present invention can afford crops with improved resistance to various environmental stresses, such as, facilitation of metabolism, promotion of photosynthesis, promotion of growth and/or promotion of root development.

1. An amino acid fertilizer composition manufactured by the steps comprising:
1) separating and producing a crude collagen peptide solution by the hydrolysis of protein from 100 wt part of dried and crushed bovine skin, pig skin or bovine bone using both pH 1.0-3.0 acid treatment and pH 12.0-14.0 alkali treatment;
2) hydrolyzing and decomposing the obtained crude collagen peptide solution into the amino acid level using both 0.01-0.5 wt part of collagen specific protease produced from Bacillus sp. variant Iren-101 and 0.001-0.1 wt part of at least one protease selected from serine endo-protease or exo-peptidase; and
3) obtaining a liquid phase amino acid fertilizer by adding 0.001-0.1 wt part of at least one water soluble inorganic salt selected from potassium, calcium, magnesium, iron or phosphate to the neutralized amino acid solution, after neutralizing the obtained amino acid solution in step 2).

2. The amino acid fertilizer composition according to claim 1, wherein the total amount of amino acid in said fertilizer composition is 38-50 wt %, and major amino acid 4.5 wt % of gultamic acid, 2.5-3.5 wt % of arginine, 1.8-2.5 wt % of aspartic acid, 2.5-3.5 wt % of leucine, 1.0-1.5 wt % of valine and 1.0-1.5 wt % of isoleucine of total fertilizer composition.

3. The amino acid fertilizer composition according to claim 1, wherein the Bacillus sp. variant Iren-101 is obtained by following isolating steps comprising:
1) selecting the strain of Bacillus sp. alive in soil showing the highest collagen assimilation property;
2) mutating the gene of the strain with N-Methyl-N′-nitro-N-nitrosoguanidine (NTG) for selecting the variant showing the highest collagen assimilation property; and
3) isolating the Bacillus sp. variant Iren-101 after more than 3 times subculturing.

4. The amino acid fertilizer composition according to claim 1, wherein a strawberry plant administered with 30-100 ppm of said amino acid fertilizer composition increases 20-30% the number of fruit of the strawberry plant, 5-10% the weight of the fruit, 10-20% the volume of the fruit and 5-10% the sugar content of the fruit compared to those of a non-administered strawberry plant.

5. The amino acid fertilizer composition according to claim 1, wherein a lettuce plant administered with 5-50 ppm of said amino acid fertilizer composition increases 25-40% the leaf length of the lettuce plant, 10-20% the leaf width and 20-30% the chlorophyll index compared to those of a non-administered lettuce plant.

6. The amino acid fertilizer composition according to claim 2, wherein the aspartic acid and glutamic acid in said amino acid fertilizer composition is effective for overcoming a physiological disorder caused by cold weather and lack of sunshine to crops, while the glycine, the alanine and the leucine increase a sugar content of the crops and the arginine improves the pathogen microbe resistance of the crops.
